(19) United States
(12) Patent Application Publication
(10) Pub. No.: US 2003/0143146 A1
(43) Pub. Date: Jul. 31, 2003

(54) Method for separating ammoniac

(76) Inventor: Hermann Luyken, Ludwigshafen (DE)

Correspondence Address: KEIL & WEINKAUF, 1350 CONNECTICUT AVENUE, N.W., WASHINGTON, DC, 20036, US

(21) Appl. No.: 10/332,585

(22) PCT Filed: Jul. 10, 2001
(86) PCT No.: PCT/EP01/07945

(30) Foreign Application Priority Data
Jul. 11, 2000 (DE) 100 33 518.7

(51) Int. Cl. C01C 1/00; C07D 201/16

(52) U.S. Cl. 423/352; 540/540

(57) ABSTRACT

A process is provided for the separation of ammonia (I) from mixtures (II) containing ammonia (I) and an amide (IV) selected from the group consisting of a lactam (IVa), an oligomer (IVb) and a polymer (IVc) with amide groups in the main chain, said amide (IV) having been obtained by reacting educts (III), selected from the group consisting of nitrites (IIIa), amines (IIIb), amino nitrites (IIIc) and amino amides (IIId), with water, wherein

a) the educt (III) is reacted with water in the liquid phase, in the presence of an organic liquid diluent (V), to give a mixture (II) containing the amide (IV) and the ammonia (I), the diluent (V) exhibiting a miscibility gap with water under certain quantity, pressure and temperature conditions,

b) the mixture (II) is converted under quantity, pressure and temperature conditions such that the diluent (V) and the water are in liquid form and exhibit a miscibility gap, to give a two-phase system consisting of a phase (VII) containing a higher proportion of diluent (V) than water, and a phase (VIII) containing a higher proportion of water than diluent (V),

c) the phase (VII) is separated from the phase (VIII),

d) all or part of the ammonia present in the phase (VII) is separated off by extraction (a) with a water-containing mixture (IX) to give an aqueous mixture (X) containing the ammonia which has been separated off, and a mixture (XI) containing less ammonia than the phase (VII), and

e) the diluent (V), any residual ammonia and any by-products selected from the group consisting of low-boiling components, high-boiling components and unreacted compounds (III) are separated from the mixture (XI) to give the amide (IV).

The present invention relates to a process for the separation of ammonia (I) from mixtures (II) obtainable by converting educts (III), selected from the group consisting of nitrites (IIIa), amines (IIIb), amino nitrites (IIIc) and amino amides (IIId), to amides (IV), wherein

a) the educt (III) is reacted with water in the liquid phase, in the presence of an organic liquid diluent (V), to give a mixture (II) containing the amide (IV), the diluent (V) exhibiting a miscibility gap with water under certain quantity, pressure and temperature conditions,

b) the mixture (II) is converted under quantity, pressure and temperature conditions such that the diluent (V) and the water are in liquid form and exhibit a miscibility gap, to give a two-phase system consisting of a phase (VII) containing a higher proportion of diluent (V) than water, and a phase (VIII) containing a higher proportion of water than diluent (V),

c) the phase (VII) is separated from the phase (VIII),

d) all or part of the ammonia present in the phase (VII) is separated off by extraction (a) with a water-containing mixture (IX) to give an aqueous mixture (X) containing the ammonia which has been separated off, and a mixture (XI) containing less ammonia than the phase (VII), and

e) the diluent (V), any residual ammonia and any by-products selected from the group consisting of low-boiling components, high-boiling components and unreacted compounds (III) are separated from the mixture (XI) to give the amide (IV).

Processes for the preparation of amides, such as cyclic lactams, by reacting omega-aminocarboxylic acid derivatives, for example the preparation of caprolactam from 6-aminocapronitrile, with water in the presence of a heterogeneous catalyst and an organic liquid diluent in the liquid phase, are generally known.

Thus WO 95/14665 and WO 95/14664 disclose the reaction of 6-aminocapronitrile in the liquid phase with water, in the presence of heterogeneous catalysts and a solvent, to give caprolactam and ammonia. The highest caprolactam yields (86 to 94%) are achieved with titanium dioxide as catalyst and ethanol as solvent. The caprolactam yields were determined only by gas chromatography in said patent documents; the work-up of the reactor discharges to crude and/or pure caprolactam is not described.

In Example 1c), WO 97/23454 describes the reaction of 6-aminocapronitrile with water in the presence of titanium dioxide and ethanol. Caprolactam was obtained from the reactor discharge by fractional distillation in a yield of 80%.

The disadvantage of said conversion of 6-aminocapronitrile to caprolactam in the presence of ethanol is the high energy consumption associated with the separation of ammonia from dilute solutions.

It is therefore an object of the present invention to provide a process which enables ammonia to be separated in a technically simple and economic manner from mixtures (II) obtainable in the conversion of educts (III) to amides (IV), and which also minimizes the energy expenditure associated with the work-up.

We have found that this object is achieved by the process defined at the outset.

According to the invention, the educts (III) are selected from the group consisting of nitriles (IIIa), amines (IIIb), amino nitriles (IIIc) and amino amides (IIId).

Suitable nitriles (IIIa) are advantageously organic compounds having one or more, such as two, three or four, preferably two, nitrile groups, i.e. preferably dinitriles, or mixtures of such compounds.

In principle, any dinitriles can be used, either individually or in a mixture. Alpha,omega-dinitriles are preferred and, of these, alpha,omega-alkylene dinitriles having from 3 to 14 C atoms or, preferably, from 3 to 12 C atoms in the alkylene radical, or an aromatic C₈-C₁₂ dinitrile such as phthalodinitrile, isophthalodinitrile or terephthalodinitrile, or a C₅-C₈ cycloalkane dinitrile such as cyclohexane dinitrile, are used in particular.

The alpha,omega-dinitriles used are preferably linear, the alkylene radical (—CH₂—)ₙ containing preferably from 2 to 14 C atoms and particularly preferably from 3 to 12 C atoms, such as ethane-1,2-dinitrile (succinic acid dinitrile), propane-1,3-dinitrile (glutaric acid dinitrile), butane-1,4-dinitrile (adipodinitrile), pentane-1,5-dinitrile (pimelic acid dinitrile), hexane-1,6-dinitrile (suberic acid dinitrile), heptane-1,7-dinitrile (azelaic acid dinitrile), octane-1,8-dinitrile (sebacic acid dinitrile), nonane-1,9-dinitrile and decane-1,10-dinitrile, particularly preferably adipodinitrile.

Adipodinitrile can be obtained by the double hydrocyanation of butadiene according to methods known per se.

Of course, it is also possible to use mixtures of several nitriles having the same number or a different number of nitrile groups, especially several dinitriles.

If desired, it is also possible to use dinitriles derived from branched alkylenes, arylenes or alkylarylenes.

Suitable amines (IIIb) are advantageously organic compounds having one or more, such as two, three or four, preferably two, amino groups, i.e. preferably diamines, or mixtures of such compounds.

In principle, any diamines can be used, either individually or in a mixture, such as aromatic amines, for example 1,4-phenylenediamine or 4,4′-diaminodiphenylpropane, or aliphatic amines. Alpha,omega-diamines are preferred and, of these, alpha,omega-alkylenediamines having from 3 to 14 C atoms or, preferably, from 3 to 10 C atoms in the alkylene radical, or alkylaryldiamines having from 9 to 14 C atoms in the alkyl radical, are used in particular, preference being given to those which contain an alkylene group having at least one C atom between the aromatic unit and the two amino groups, such as p-xylylenediamine or, preferably, m-xylylenediamine.

The alpha,omega-diamines used are preferably linear, the alkylene radical (—CH₂—)ₙ having preferably from 3 to 14 C atoms and particularly preferably from 3 to 10 C atoms, such as 1,3-diaminopropane, 1,4-diaminobutane, 1,5-diaminopentane, 1,6-diaminohexane (hexamethylenediamine, HMD), 1,7-diaminoheptane, 1,8-diaminooctane, 1,9-diaminononane and 1,10-diaminodecane, particularly preferably hexamethylenediamine.

Hexamethylenediamine can be obtained by the double catalytic hydrogenation of the nitrile groups of adipodinitrile according to methods known per se.

Of course, it is also possible to use mixtures of several diamines.

If desired, it is also possible to use diamines derived from branched alkylenes, arylenes or alkylarylenes, such as 2-methyl-1,5-diaminopentane.

Suitable amino nitriles (IIIc) are advantageously organic compounds having one or more, such as two, three or four, amino groups, preferably one amino group, and one or more, such as two, three or four, nitrile groups, preferably one nitrile group, i.e. preferably monoamino mononitriles (“aminocarboxylic acid nitriles”), or mixtures of such compounds.

Omega-aminocarboxylic acid nitriles are preferred and, of these, omega-aminocarboxylic acid nitriles having from 3 to 12 C atoms or, preferably, from 3 to 9 C atoms in the alkylene radical, or aminoalkylarylcarboxylic acid nitriles having from 7 to 13 C atoms in the alkylene radical, are used in particular, preference being given to those which contain an alkylene group having at least one C atom between the aromatic unit and the amino and nitrile groups. Particularly preferred aminoalkylarylcarboxylic acid nitriles are those in which the amino and nitrile groups are in the 1,4-positions relative to one another.

The omega-aminocarboxylic acid nitriles used are preferably linear, the alkylene radical (—CH₂—)ₙ having preferably from 3 to 14 C atoms and particularly preferably from 3 to 9 C atoms, such as 3-amino-1-nitrilopropane, 4-amino-1-nitrilobutane, 5-amino-1-nitrilopentane (6-aminocapronitrile), 6-amino-1-nitrilohexane, 7-amino-1-nitriloheptane, 8-amine-1-nitrilooctane [sic] and 9-amino-1-nitrilononane, particularly preferably 6-aminocapronitrile.

6-Aminocapronitrile can be obtained by the simple catalytic hydrogenation of one of the nitrile groups of adipodinitrile according to methods known per se.

Of course, it is also possible to use mixtures of several aminocarboxylic acid nitriles.

If desired, it is also possible to use aminocarboxylic acid nitriles derived from branched alkylenes, arylenes or alkylarylenes.

Suitable amino amides (IIId) are advantageously organic compounds having one or more, such as two, three or four, amino groups, preferably one amino group, and one or more, such as two, three or four, carboxamide groups (—CONH₂), preferably one carboxamide group, i.e. preferably monoamino monoamides “(aminocarboxamides”), or mixtures of such compounds.

Omega-aminocarboxamides are preferred and, of these, omega-aminocarboxamides having from 3 to 12 C atoms or, preferably, from 3 to 9 C atoms in the alkylene radical, or aminoalkylarylcarboxamides having from 7 to 13 C atoms in the alkylene radical, are used in particular, preference being given to those which contain an alkylene group having at least one C atom between the aromatic unit and the amino and carboxamide groups. Particularly preferred aminoalkylarylcarboxamides are those in which the amino and carboxamide groups are in the 1,4-positions relative to one another.

The omega-aminocarboxamides used are preferably linear, the alkylene radical (—CH₂—)ₙ having preferably from 3 to 14 C atoms and particularly preferably from 3 to 9 C atoms, such as 3-amino-1-carboxamidopropane, 4-amino-1-carboxamidobutane, 5-amino-1-carboxamidopentane (6-aminohexanamide), 6-amino-1-carboxamidohexane, 7-amino-1-carboxamidoheptane, 8-amine-1-carboxamidooctane [sic] and 9-amino-1-carboxamidononane, particularly preferably 6-aminohexanamide.

6-Aminohexanamide can be obtained by partial hydrolysis of the nitrile group of 6-aminocapronitrile according to methods known per se.

Of course, it is also possible to use mixtures of several aminocarboxamides.

If desired, it is also possible to use aminocarboxamides derived from branched alkylenes, arylenes or alkylarylenes.

It is also possible to use mixtures of compounds (IIIa), (IIIb), (IIIc) and (IIId).

In addition to the compounds (IIIa), (IIIb), (IIIc) and (IIId), the educt (III) can contain other compounds which have functional groups capable of forming the amide groups of (IV), such as carboxylic acid groups, carboxylic acid ester groups or lactams, for example adipic acid or caprolactam.

If the educt (III) contains a nitrile (IIIa) and an amine (IIIb), for example if the educt (III) contains adipodinitrile and hexamethylenediamine in the presence or absence of compounds (IIIc) and/or (IIId), the molar ratio of the nitrile groups of (IIIa) involved in forming the amide groups of (IV) to the amine groups of (IIIb) involved in forming the amide groups of (IV) is advantageously between 0.8 and 1.2, preferably between 0.95 and 1.05 and particularly preferably between 0.98 and 1.02 (equimolar).

Step a) of the process according to the invention yields an amide (IV) selected from the group consisting of a lactam (IVa), an oligomer (IVb) and a polymer (IVc) with amide groups in the main chain.

Lactams (IVa) can advantageously be obtained from educts capable of forming an internal amide group with themselves, preferably from (IIIc) and (IIId). The structure of the lactams (IVa) is then related directly to the structure of the educts (III).

In terms of the present invention, oligomers (IVb) are understood as meaning compounds which result from the coupling of a few molecules, such as two, three, four, five or six molecules, selected from the group comprising the compounds used as the educt (III), via amide functional groups, such as dimers, trimers, tetramers, pentamers or hexamers of 6-aminocapronitrile, 6-aminohexanamide or an adipodinitrile/hexamethylenediamine mixture, or mixtures thereof.

In terms of the present invention, polymers (IVc) are understood as meaning high-molecular compounds which have recurring amide groups (—CONH—) in the main chain, for example polycaprolactam (nylon 6) or poly(hexamethyleneammonium adipate) (nylon 6,6).

In step a) of the process according to the invention, the above-described educt (III) is reacted with water in the liquid phase, preferably in a homogeneous liquid phase, advantageously in the presence of a heterogeneous catalyst and an organic liquid diluent (V), to give a mixture (II) containing an amide (IV), said diluent (V) exhibiting a miscibility gap with water under certain quantity, pressure and temperature conditions.

Suitable heterogeneous catalysts are acidic, basic or amphoteric oxides of the elements of main group II, III or IV of the periodic table, such as calcium oxide, magnesium oxide, boron oxide, aluminum oxide, tin oxide or silicon dioxide in the form of pyrogenic silicon dioxide, silica gel, kieselguhr, quartz or mixtures thereof, and also oxides of metals of subgroups II to VI of the periodic table, such as amorphous titanium dioxide in the form of anatase or rutile, zirconium dioxide, manganese oxide or mixtures thereof. It is also possible to use lanthanide and actinide oxides such as cerium oxide, thorium oxide, praseodymium oxide, samarium oxide, a rare earth mixed oxide or mixtures thereof with the abovementioned oxides. Examples of other possible catalysts are:

vanadium oxide, barium oxide, zinc oxide, niobium oxide, iron oxide, chromium oxide, molybdenum oxide, tungsten oxide or mixtures thereof. Mixtures of said oxides with one another are also possible. Some sulfides, selenides and tellurides, such as zinc telluride, tin selenide, molybdenum sulfide, tungsten sulfide and the sulfides of nickel, zinc and chromium, can also be used.

The abovementioned compounds can be doped with, or contain, compounds of main groups I and VII of the periodic table.

Other suitable catalysts which may be mentioned are zeolites, phosphates and heteropolyacids, as well as acidic and alkaline ion exchangers like Nafion.

Preferred catalysts are titanium oxide, aluminum oxide, cerium oxide and zirconium dioxide, particularly preferred catalysts being titanium dioxides such as those disclosed e.g. in WO 96/36600. The preparation of such catalysts as pellets is described for example in WO 99/11613, WO 99/11614 and WO 99/11615.

Suitable diluents (V) are C₄ to C₉ alkanols such as n-butanol, i-butanol or n-pentanol, preferably aliphatic hydrocarbons such as n-hexane, cycloaliphatic hydrocarbons such as cyclopentane or cyclohexane, and particularly preferably aromatic hydrocarbons such as benzene, toluene, o-xylene, m-xylene, p-xylene, ethylbenzene, i-propylbenzene or di-i-propylbenzene, especially benzene, toluene, o-xylene, m-xylene, p-xylene or ethylbenzene, as well as mixtures of such compounds, for example petroleum ethers. The hydrocarbons can carry functional groups such as halogens, for example chlorine, as in chlorobenzene.

In the reaction of step a), at least 1 mol, preferably 2 to 100 mol and particularly preferably 2 to 10 mol of water should generally be used per mol of compound (III).

In step a), the proportion of compound (III), based on the sum of the starting components, namely compound (III), water and diluent (V), is advantageously 0.1 to 50% by weight, preferably 1 to 30% by weight and particularly preferably 2 to 20% by weight.

The reaction can advantageously be carried out in the liquid phase at temperatures generally of 140 to 320° C., preferably of 180 to 300° C. and particularly preferably of 200 to 280° C. The pressure should generally range from 1 to 250 bar and preferably from 5 to 150 bar.

The preferred pressure and temperature conditions here are those under which the reaction mixture is in the form of a single homogeneous liquid phase.

The catalyst loadings generally range from 0.05 to 5 kg, preferably from 0.1 to 2 kg and particularly preferably from 0.2to 1 kg of reaction mixture per catalyst volume per hour.

The reaction of step a) yields a mixture (II) containing an amide (IV), ammonia (I) and optionally by-products selected from the group consisting of low-boiling components, high-boiling components and unreacted compound (III).

In terms of the present invention, low-boiling components are understood as meaning compounds boiling below the amide (IV) and high-boiling components (VII) are understood as meaning compounds boiling above the amide (IV).

According to the invention, in step b), the mixture (II) is converted under quantity, pressure and temperature conditions such that the diluent (V) and the water are in liquid form and exhibit a miscibility gap, to give a two-phase system comprising a phase (VII) in which the proportion of diluent (V) is greater than that of water, and a phase (VIII) in which the proportion of water is greater than that of diluent (V).

Preferred quantity, pressure and temperature conditions are those under which the constituents of the mixture (II) are in completely liquid form in the phases (VII) and (VIII), i.e. under which no solids precipitate out.

If step a) has been carried out in a homogeneous liquid phase, it is generally possible to separate the mixture (II) into the two phases (VII) and (VIII) by choosing a suitable temperature. A further possibility is to choose suitable proportions, for instance by adding diluent (V) or, preferably, water.

According to the invention, the phase (VII) and the phase (VIII) are then separated in step c).

The phase separation can be effected in a manner known per se in apparatuses described for such purposes, such as those known e.g. from: Ullmann's Encyclopedia of Industrial Chemistry, vol. B3, 5th ed., VCH Verlagsgesellschaft, Weinheim, 1988, pages 6-14 to 6-22, like decanters, cyclones or centrifuges.

The optimum apparatuses and process conditions for the phase separation can easily be determined by a few simple preliminary experiments.

According to the invention, in step d), all or part of the ammonia present in the phase (VII) are [sic] separated off by extraction (a) with a water-containing mixture (IX) to give an aqueous mixture (X) containing the ammonia which has been separated off, and a mixture (XI) containing less ammonia than the phase (VII).

The mixture (IX) used can advantageously be water, wholly or partially a mixture (XIII) defined below, wholly or partially a mixture (XIV) defined below whose water content is greater than that of the mixture (XIII), or mixtures thereof.

The extraction (a) can be effected in a manner known per se in apparatuses described for such purposes, such as those known e.g. from: Ullmann's Encyclopedia of Industrial Chemistry, vol. B3, 5th ed., VCH Verlagsgesellschaft, Weinheim, 1988, pages 6-14 to 6-22, like sieve-[lacuna] or packed columns, pulsating or non-pulsating, or mixer-settlers.

The optimum apparatuses and process conditions for the extraction (a) can easily be determined by a few simple preliminary experiments.

According to the invention, in step e), the diluent (V), any residual ammonia and any by-products selected from the group consisting of low-boiling components, high-boiling components and unreacted compound (III) are separated from the mixture (XI) to give the amide (IV).

In terms of the present invention, low-boiling components are understood as meaning compounds boiling below the amide (IV) and high-boiling components are understood as meaning compounds boiling above the amide (IV).

This work-up can advantageously be effected by fractional distillation in one or more, such as 2 or 3, distillation apparatuses.

Suitable apparatuses are those conventionally used for distillation, for example those described in: Kirk-Othmer, Encyclopedia of Chemical Technology, 3rd ed., vol. 7, John Wiley & Sons, New York, 1979, pages 870-881, such as sieve-plate columns, bubble-cap columns or packed columns.

Advantageously, all or part of the ammonia can be separated from the phase (VIII), preferably from the phase (VIII) and the mixture (X) together, by distillation (b1) or rectification (b2) to give a mixture (XII) containing the bulk of the ammonia, and a mixture (XIII) in which the ammonia content is less than that of the phase (VIII).

A suitable procedure is preferably a distillative separation (b1) or (b2) of the ammonia at a pressure of less than 8 bar absolute, the ammonia being withdrawn especially in the vapor state.

This work-up can advantageously be effected by fractional distillation in one or more, such as 2 or 3, distillation apparatuses.

Suitable apparatuses are those conventionally used for distillation, for example those described in: Kirk-Othmer, Encyclopedia of Chemical Technology, 3rd ed., vol. 7, John Wiley & Sons, New York, 1979, pages 870-881, such as sieve-plate columns, bubble-cap columns or packed columns, especially a column with a side discharge.

In the case of a column with a side discharge, a mixture (XIV) can be obtained at a side discharge of the device used in the distillation (b1) or the rectification (b2).

The ammonia withdrawn in the vapor state can advantageously be subjected to a treatment (c) with an alkali (XV) to give a purified ammonia (XVI). Suitable alkalis (XV) are compounds which give a basic reaction, preferably oxides and hydroxides and particularly preferably those of main groups I and II, such as sodium hydroxide.

This work-up can advantageously be effected by scrubbing in one or more, such as 2 or 3, apparatuses through which the ammonia (XII) and the scrubbing agent (XV) are advantageously passed in countercurrent.

Suitable apparatuses are those conventionally used for scrubbing, for example those described in: Kirk-Othmer, Encyclopedia of Chemical Technology, 3rd ed., vol. 7, John Wiley & Sons, New York, 1979, pages 870-881, such as sieve-plate columns, bubble-cap columns, packed columns, Venturi scrubbers or spray columns.

In one advantageous embodiment, the mixture (XII) or the ammonia (XVI) can be absorbed in water, (d), to give an aqueous mixture (XVII) containing ammonia.

In another advantageous embodiment, the mixture (XII) or the ammonia (XVI) can be compressed to a higher pressure to give a mixture (XVIII).

The mixture (XII) or the mixture (XIII) can be distilled at a pressure of more than 8 bar absolute to give a mixture (XIX) containing less water and less diluent (V) than the mixture (XVIII), and a mixture (XX) containing less ammonia than the mixture (XVIII).

All or part of the mixture (XX) can advantageously be used in the absorption (d).

The diluent (V) can advantageously be separated from the mixture (XX) and recycled into step a) of the process according to the invention.

In another advantageous embodiment, all or part of the mixture (XIII) can be recycled into step a) of the process according to the invention.

The amides (IV) obtainable by the process according to the invention are valuable intermediates in the preparation of industrially important polymers, especially polyamides. Such polyamides, as well as the polymer (IVc), can be used for the production of fibers, sheets and moldings in a manner known per se.

We claim:

1. A process for the separation of ammonia (I) from mixtures (II) containing ammonia (I) and an amide (IV) selected from the group consisting of a lactam (IVa), an oligomer (IVb) and a polymer (IVc) with amide groups in the main chain, said amide (IV) having been obtained by reacting educts (III), selected from the group consisting of nitriles (IIIa), amines (IIIb), amino nitriles (IIIc) and amino amides (IIId), with water, wherein
a) the educt (III) is reacted with water in the liquid phase, in the presence of an organic liquid diluent (V), to give a mixture (II) containing the amide (IV) and the ammonia (I), the diluent (V) exhibiting a miscibility gap with water under certain quantity, pressure and temperature conditions,
b) the mixture (II) is converted under quantity, pressure and temperature conditions such that the diluent (V) and the water are in liquid form and exhibit a miscibility gap, to give a two-phase system consisting of a phase (VII) containing a higher proportion of diluent (V) than water, and a phase (VIII) containing a higher proportion of water than diluent (V),
c) the phase (VII) is separated from the phase (VIII),
d) all or part of the ammonia present in the phase (VII) is separated off by extraction (a) with a water-containing mixture (IX) to give an aqueous mixture (X) containing the ammonia which has been separated off, and a mixture (XI) containing less ammonia than the phase (VII), and
e) the diluent (V), any residual ammonia and any by-products selected from the group consisting of low-boiling components, high-boiling components and unreacted compounds (III) are separated from the mixture (XI) to give the amide (IV).

2. A process as claimed in claim 1 wherein all or part of the ammonia is separated from the phase (VIII) by distillation (b1) or rectification (b2) to give a mixture (XII) containing essentially ammonia, and a mixture (XIII) in which the ammonia content is less than that of the phase (VIII).

3. A process as claimed in claim 1 or 2 wherein the phase (VIII) and the mixture (X) are worked up together in the distillation (b1) or the rectification (b2) and the ammonia is separated off.

4. A process as claimed in any of claims 1 to 3 wherein all or part of the mixture (XIII) is used as the aqueous mixture (IX).

5. A process as claimed in any of claims 1 to 4 wherein a mixture (XIV) in which the water content is greater than that of the mixture (XIII) is used as the aqueous mixture (IX).

6. A process as claimed in claim 5 wherein the mixture (XIV) is obtained at a side discharge of the device used in the distillation (b1) or the rectification (b2).

7. A process as claimed in any of claims 1 to 6 wherein all or part of the mixture (XIII) is recycled into the reactor for synthesizing the amide (IV) from the educt (III).

8. A process as claimed in any of claims 2 to 7 wherein the distillative separation (b1) or (b2) of the ammonia is carried out at a pressure of less than 8 bar absolute and the ammonia is withdrawn in the vapor state.

9. A process as claimed in claim 8 wherein the ammonia withdrawn in the vapor state is subjected to a treatment (c) with an alkali (XV) to give a purified ammonia (XVI).

10. A process as claimed in claim 9 wherein NaOH is used as the alkali (XV).

11. A process as claimed in any of claims 1 to 10 wherein the mixture (XII) or the ammonia (XVI) is absorbed in water, (d), to give an aqueous mixture (XVII) containing ammonia.

12. A process as claimed in any of claims 1 to 10 wherein the mixture (XII) or the ammonia (XVI) is compressed to a higher pressure to give a mixture (XVIII).

13. A process as claimed in claim 11 or 12 wherein the mixture (XVII) or the mixture (XVIII) is distilled at a pressure of more than 8 bar absolute to give a mixture (XIX) containing less water and less diluent (V) than the mixture (XVIII), and a mixture (XX) containing less ammonia than the mixture (XVII) or the mixture (XVIII).

14. A process as claimed in claim 11 or 13 wherein all or part of the mixture (XX) is used for the absorption (d).

15. A process as claimed in claim 13 or 14 wherein the diluent (V) is separated from the mixture (XX) and recycled into the synthesis of the amide (IV) from the educt (III).

16. A process as claimed in any of claims 1 to 15 wherein 6-aminocapronitrile is used as the amino nitrile (IIIc).

17. A process as claimed in any of claims 1 to 16 wherein adipodinitrile is used as the nitrile (IIIa).

18. A process as claimed in any of claims 1 to 17 wherein hexamethylenediamine is used as the amine (IIIb).

19. A process as claimed in any of claims 1 to 18 wherein a diluent (V) selected from the group consisting of ethylbenzene, benzene, toluene, o-xylene, m-xylene and p-xylene is used.
